(19) United States
(12) Patent Application Publication
(10) Pub. No.: US 2025/0058338 A1
(43) Pub. Date: Feb. 20, 2025

(54) OPTICAL ATOMIZER, SPRAYING METHOD USING THE SAME, AND MANUFACTURING METHOD OF THE SAME

(71) Applicant: UIF (University Industry Foundation), Yonsei University, Seoul (KR)

(72) Inventors: Kyunghwan OH, Seoul (KR); Meongrae Kim, Seoul (KR); Hyeonwoo Lee, Seoul (KR)

(73) Assignee: UIF (University Industry Foundation), Yonsei University, Seoul (KR)

(21) Appl. No.: 18/806,276

(22) Filed: Aug. 15, 2024

(30) Foreign Application Priority Data
Aug. 17, 2023 (KR) 10-2023-0107563

(51) Int. Cl. B05B 7/22; B23K 26/352

(52) U.S. Cl. CPC B05B 7/228; B23K 26/352

(57) ABSTRACT

Disclosed is an optical atomizer. The optical atomizer according to the present disclosure includes a laser transmitter unit configured to transmit a laser beam oscillated by a laser oscillator to one side, and a sprayer unit connected to the laser transmitter unit, and configured to heat a liquid for spraying received in the sprayer unit by the laser beam transmitted from the laser transmitter unit and spray the evaporated liquid for spraying to outside.

### CROSS-REFERENCE TO RELATED APPLICATION

This application claims priority to Korean Patent Application No. 10-2023-0107563 filed on Aug. 17, 2023, and all the benefits accruing therefrom under 35 U.S.C. § 119, the contents of which in its entirety are herein incorporated by reference.

### BACKGROUND

### 1. Field

The present disclosure relates to an optical atomizer, a spraying method using the optical atomizer and a method for manufacturing the optical atomizer, and more particularly, to an optical atomizer for spraying a liquid using an optical fiber and a laser, a spraying method using the optical atomizer and a method for manufacturing the optical atomizer.

### 2. Description of the Related Art

In general, a liquid ejector is widely used in different sections across all fields, and has a wide range of applications and comes in different types in the medical, mechanical engineering, electronic and chemical field.

The liquid ejector is a device that conveys a surrounding fluid to another location by pumping water, air or vapor having pressure from an outlet at a high speed.

Recently, many liquid ejectors for drug delivery, printing and particle delivery are being developed, and these liquid ejectors are designed to spray liquids using ultrasound, aerodynamics, electricity, etc.

In particular, in the case of drug delivery, a variety of drug delivery systems have been used for parenteral administration of drugs for treatment into patients' bodies. The drug delivery systems use syringes the most commonly, and the syringes are a fearful thing that threatens patients due to pain when injecting, and have unavoidable risks of infection caused by needle stick injuries.

To solve this problem, drug delivery technologies such as needleless injection are being developed. That is, drugs are directly injected into the human body through the living tissues by high-speed microjet spraying, instead of directly poking the skin tissues with needles.

For high-speed microjet spraying, it is necessary to spray drugs to the outside (i.e., skin) precisely and strongly. A variety of spraying methods have been developed since 1930's, and recently a variety of spraying methods have been developed, for example, spraying methods using piezoelectric ceramic devices, spraying methods using Lorentz force through shock waves induced by a laser beam applied to an aluminum foil.

Additionally, technologies using springs and high pressure gases have been developed, and drugs are directly injected into the human body through the living tissues by spraying microdroplets or streams of drugs using springs and high pressure gases.

However, the existing liquid ejectors require high production costs due to their intricate structure, and cannot use high output sources, resulting in low utilization.

### RELATED LITERATURES

### Patent Literature

Korean Patent Publication No. 10-2018-0040994 (2018.04.23.)

### SUMMARY

The present disclosure is directed to providing an optical atomizer with simple design of the entire device and high utilization by the use of high power source, a spraying method using the optical atomizer and a method for manufacturing the optical atomizer.

According to an aspect of the present disclosure, there is provided an optical atomizer including a laser transmitter unit configured to transmit a laser beam oscillated by a laser oscillator to one side; and a sprayer unit connected to the laser transmitter unit, and configured to heat a liquid for spraying received in the sprayer unit by the laser beam transmitted from the laser transmitter unit and spray the evaporated liquid for spraying to outside.

The sprayer unit may include a spray body having an end portion joined to the laser transmitter unit, and an opposite end portion having an opening of a hollow groove in which the liquid for spraying is received; and a metal foil deposited on the opposite end portion of the spray body, and having a spray hole in communication with the hollow groove.

The spray body may be made of a coreless Hollow Optical Fiber (HOF).

The liquid for spraying may include a high viscosity liquid.

The liquid for spraying may include any one selected from triethylene glycol, glycerol and oleic acid.

The metal foil may be deposited on the spray body by a sputtering process.

The metal foil may be made of titanium.

A bottom area of the hollow groove may be formed with a smaller diameter toward the laser transmitter unit.

The sprayer unit may further include a cladding for sprayer surrounding an outer surface of the spray body.

The cladding for sprayer may be formed by applying a low-refractive-index optical fiber coating agent to the outer surface of the spray body.

The spray body may have a long-period fiber grating to excite the laser beam transmitted from the laser transmitter unit in a cladding mode.

The long-period fiber grating may be formed by ultraviolet laser beam irradiation to the spray body.

According to another aspect of the present disclosure, there is provided a spraying method using an optical atomizer, including a laser transmission step of transmitting a laser beam oscillated by a laser oscillator to a spray body having a hollow groove in which a liquid for spraying is received; a heating and spraying step of heating a metal foil deposited on the spray body and having a spray hole in communication with the hollow groove by the transmitted laser beam, and spraying the liquid for spraying evaporated by the heated metal foil out of the hollow groove; and a condensation step of condensing the evaporated liquid for spraying sprayed out of the hollow groove into droplets.

The metal foil may be heated by a photothermal effect in the heating and spraying step.

The liquid for spraying may include any one selected from triethylene glycol, glycerol and oleic acid, and the metal foil may be made of titanium.

According to still another aspect of the present disclosure, there is provided a method for manufacturing an optical atomizer, including a joining step of joining a spray body having a hollow groove to a laser transmitter unit configured to transmit a laser beam oscillated by a laser oscillator to one side; a metal foil deposition step of depositing a metal foil having a spray hole in communication with the hollow groove on the spray body; and a liquid injection step of injecting a liquid for spraying into the hollow groove.

The metal foil may be deposited on the spray body by a sputtering process.

The metal foil may be made of titanium.

The liquid for spraying may be injected into the hollow groove through a capillary phenomenon in the liquid injection step.

The method for manufacturing the optical atomizer may further include, after the metal foil deposition step, a cladding formation step of forming a cladding for sprayer surrounding an outer surface of the spray body.

The cladding formation step may include applying a low-refractive-index optical fiber coating agent to an outer surface of the spray body to form the cladding for sprayer.

The method for manufacturing the optical atomizer may further include, after the metal foil deposition step, a grating formation step of forming a long-period fiber grating in the spray body to excite the laser beam transmitted from the laser transmitter unit in a cladding mode.

The grating formation step may include forming the long-period fiber grating by ultraviolet laser beam irradiation to the spray body.

The embodiments of the present disclosure include the sprayer unit configured to heat the liquid for spraying by the laser beam transmitted from the laser transmitter unit and spray the evaporated liquid for spraying to the outside, and thus the design of the entire device is simple and the high power source is used, so there is a high utilization advantage.

### BRIEF DESCRIPTION OF THE DRAWINGS

FIG. 1 is a cross-sectional view showing an optical atomizer according to a first embodiment of the present disclosure.

FIG. 2 is a flowchart showing a spraying method using the optical atomizer of FIG. 1.

FIG. 3 is a flowchart showing a method for manufacturing the optical atomizer of FIG. 1.

FIG. 4 is a cross-sectional view showing an optical atomizer according to a second embodiment of the present disclosure.

FIG. 5 is a flowchart showing a method for manufacturing the optical atomizer of FIG. 4.

FIG. 6 is a cross-sectional view showing an optical atomizer according to a third embodiment of the present disclosure.

FIG. 7 is a flowchart showing a method for manufacturing the optical atomizer of FIG. 6.

### DETAILED DESCRIPTION

For sufficient understanding of the present disclosure, its operational advantages and objectives achieved by embodying the present disclosure, reference is made to the accompanying drawings illustrating exemplary embodiments of the present disclosure and the text matter in the accompanying drawings.

Hereinafter, the present disclosure will be described in detail by describing the exemplary embodiments of the present disclosure with reference to the accompanying drawings. However, in describing the present disclosure, a description of known functions or elements is omitted for clarity.

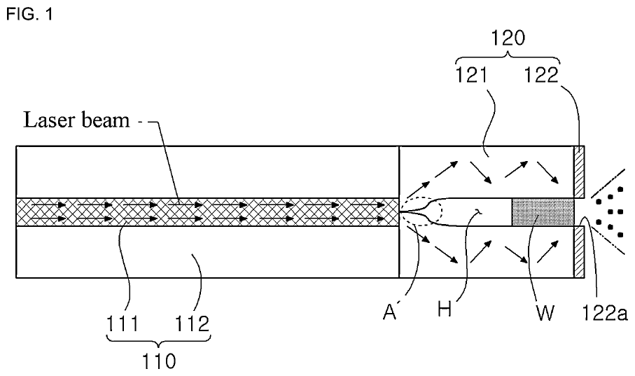

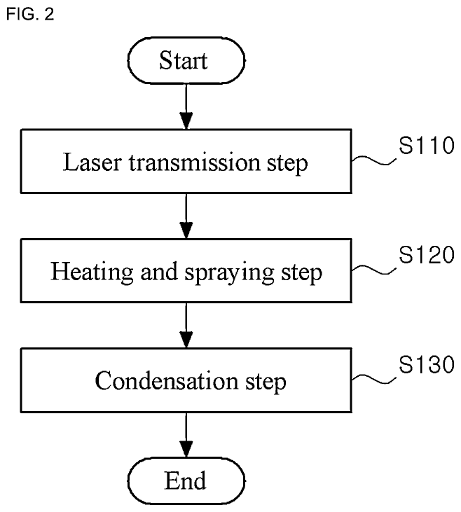

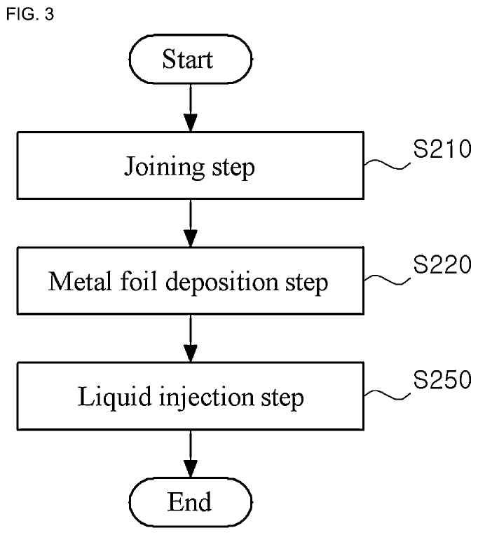

FIG. 1 is a cross-sectional view showing an optical atomizer according to a first embodiment of the present disclosure, FIG. 2 is a flowchart showing a spraying method using the optical atomizer of FIG. 1, and FIG. 3 is a flowchart showing a method for manufacturing the optical atomizer of FIG. 1.

As shown in FIG. 1 in detail, the optical atomizer according to this embodiment includes a laser transmitter unit 110 to transmit a laser beam oscillated by a laser oscillator (not shown) to one side, and a sprayer unit 120 connected to the laser transmitter unit 110 and configured to heat a liquid W for spraying received in the sprayer unit 120 by the laser beam transmitted from the laser transmitter unit 110 and spray the evaporated liquid W for spraying to the outside.

The laser oscillator (not shown) oscillates the laser beam (light amplification by stimulated emission of radiation). The laser beam is amplified through repeated stimulated emission by repeated reflection in an amplifier.

The laser oscillator (not shown) has various wavelengths, and the laser oscillator (not shown) according to this embodiment oscillates the laser beam having the wavelength of 976 nanometers (nm), and the scope of protection of the present disclosure is not limited thereto and the laser oscillator (not shown) that oscillates the laser beam of various wavelengths may be used in this embodiment.

The laser transmitter unit 110 transmits the laser beam oscillated by the laser oscillator (not shown) to one side. The laser transmitter unit 110 allows the laser beam oscillated by the laser oscillator (not shown) to pass through, and is made of a single-mode optical fiber. The laser transmitter unit 110 may be substituted by a multiple-mode optical fiber.

The laser transmitter unit 110 made of a single-mode optical fiber includes a core 111, a cladding 112 for transmitter surrounding the core 111, and a synthetic resin (not shown) coated on the cladding 112 for transmitter. The core 111 is formed with a diameter of a few micrometers. The laser beam oscillated by the laser oscillator (not shown) may move through the core 111, thereby reducing losses during movement.

The sprayer unit 120 is connected to the laser transmitter unit 110. The sprayer unit 120 heats the liquid W for spraying received in the sprayer unit 120 by the laser beam transmitted from the laser transmitter unit 110 and sprays the evaporated liquid W for spraying to the outside.

As shown in FIG. 1, the sprayer unit 120 according to this embodiment includes a spray body 121 with one end portion joined to the laser transmitter unit 110 and the other end portion having a hollow groove H in which the liquid W for spraying is received, and a metal foil 122 deposited on the other end portion of the spray body 121 and having a spray hole 122a in communication with the hollow groove H.

In this embodiment, the spray body 121 is made of a coreless Hollow Optical Fiber (HOF).

As shown in FIG. 1, one end portion of the spray body 121 is joined to the laser transmitter unit 110. In this embodiment, the spray body 121 and the laser transmitter unit 110 may be joined to each other by insulation welding.

The refractive index of the spray body 121 is higher than the refractive index of outdoor air, to focus the laser beam to the spray body 121 and direct it toward the metal foil 122.

The spray body 121 has the hollow groove H in which the liquid W for spraying is received. The spray body 121 has an opening of the hollow groove H at the other end portion. A bottom area A of the hollow groove H is formed with a smaller diameter toward the laser transmitter unit 110.

Because the spray body 121 has the hollow groove H having the bottom area A with a smaller diameter toward the laser transmitter unit 110 as described above, the laser beam transmitted from the laser transmitter unit 110 may easily reach the metal foil 122 through the spray body 121.

The liquid W for spraying is received in the hollow groove H. The liquid W for spraying includes a high viscosity liquid. In this embodiment, the liquid W for spraying may include any one of triethylene glycol, glycerol and oleic acid.

The metal foil 122 is deposited on the other end portion of the spray body 121. The spray hole 122a in communication with the hollow groove H of the spray body 121 is formed at the central area of the metal foil 122.

In this embodiment, the metal foil 122 is deposited on the other end portion of the spray body 121 by a sputtering process. The metal foil 122 may be made of titanium.

The metal foil 122 according to this embodiment absorbs the laser beam having passed through the spray body 121 to generate heat by the photothermal effect. The generated heat evaporates the liquid W for spraying received in the hollow groove H. The evaporated liquid W for spraying is condensed into droplets after it is sprayed (ejected) out of the hollow groove H.

As described above, the optical atomizer of this embodiment uses the metal foil 122, and compared to when carbon nanotubes are used, it is possible to increase the power of the laser oscillator (not shown) up to 100 milliwatts, thereby achieving a high utilization advantage (when carbon nanotubes are used, the power of the laser oscillator amounts to 19 milliwatts).

Hereinafter, a spraying method using the optical atomizer according to this embodiment will be described with reference to FIGS. 1 and 2.

The spraying method using the optical atomizer according to this embodiment includes, as shown in FIGS. 1 and 2, a laser transmission step (S110) of transmitting the laser beam oscillated by the laser oscillator (not shown) to the spray body 121 having the hollow groove H in which the liquid W for spraying is received, a heating and spraying step (S120) of heating the metal foil 122 deposited on the spray body 121 and having the spray hole 122a in communication with the hollow groove H by the transmitted laser beam, and spraying the liquid W for spraying evaporated by the heated metal foil 122 out of the hollow groove H, and a condensation step (S130) of condensing the evaporated liquid W for spraying sprayed out of the hollow groove H into droplets.

In the laser transmission step (S110), the laser beam oscillated by the laser oscillator (not shown) moves toward the metal foil 122 through the laser transmitter unit 110 and the spray body 121.

In the heating and spraying step (S120), the metal foil 122 irradiated by the laser beam is heated by the photothermal effect. The liquid W for spraying is evaporated by the heated metal foil 122 and the evaporated liquid W for spraying is sprayed (ejected) out of the hollow groove H.

In the condensation step (S130), the evaporated liquid W for spraying sprayed (ejected) from the hollow groove H to the outside through the spray hole 122a of the metal foil 122 is condensed into droplets by low outdoor temperature.

Because the optical atomizer according to this embodiment includes the sprayer unit 120 to heat the liquid W for spraying received in the sprayer unit 120 by the laser beam transmitted from the laser transmitter unit 110 and spray the evaporated liquid W for spraying to the outside, the design of the entire device is simple and the high power source can be used, so there is a high utilization advantage.

Hereinafter, a method for manufacturing the optical atomizer according to this embodiment will be described with reference to FIGS. 1 and 3.

As shown in FIG. 3, the method for manufacturing the optical atomizer includes a joining step (S210) of joining the spray body 121 having the hollow groove H to the laser transmitter unit 110 configured to transmit the laser beam oscillated by the laser oscillator (not shown) to one side, a metal foil deposition step (S220) of depositing the metal foil 122 having the spray hole 122a in communication with the hollow groove H on the spray body 121, and a liquid injection step (S250) of injecting the liquid W for spraying into the hollow groove H.

In the joining step (S210), one end portion of the spray body 121 is joined to the laser transmitter unit 110. In this embodiment, the spray body 121 and the laser transmitter unit 110 may be joined to each other by insulation welding. Subsequently, the spray body 121 is cut into a predetermined length (about 5 mm).

In the metal foil deposition step (S220), the metal foil 122 is deposited on the other end portion of the spray body 121. In this embodiment, the metal foil 122 is formed by depositing titanium on the other end portion of the spray body 121 by the sputtering process.

In the liquid injection step (S250), the liquid W for spraying is injected into the hollow groove H. In the liquid injection step (S250) according to this embodiment, the liquid W for spraying is injected into the hollow groove H through a capillary phenomenon. Because the method for manufacturing the optical atomizer according to this embodiment includes the joining step (S210) of joining the spray body 121 having the hollow groove H to the laser transmitter unit 110 configured to transmit the laser beam oscillated by the laser oscillator (not shown) to one side, the metal foil deposition step (S220) of depositing the metal foil 122 having the spray hole 122a in communication with the hollow groove H on the spray body 121, and the liquid injection step (S250) of injecting the liquid W for spraying into the hollow groove H, it may be possible to manufacture the optical atomizer in a straightforward and economically-efficient manner.

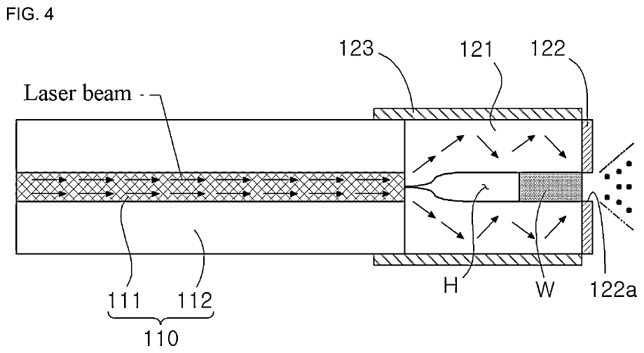

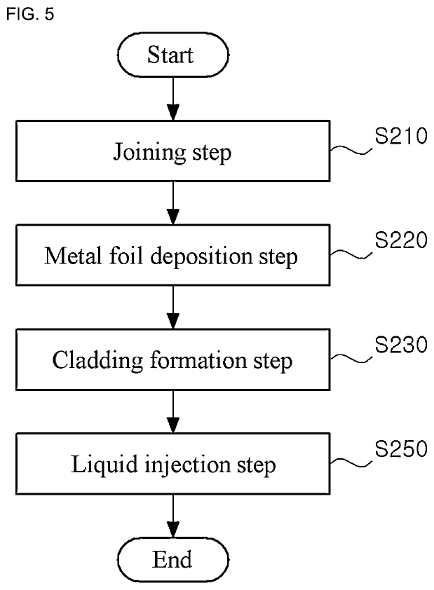

FIG. 4 is a cross-sectional view showing the optical atomizer according to a second embodiment of the present disclosure, and FIG. 5 is a flowchart showing the method for manufacturing the optical atomizer of FIG. 4.

When compared with the first embodiment, this embodiment further includes a cladding 123 for sprayer, but has the same components as the first embodiment of FIGS. 1 to 3, and hereinafter the description of the same components is omitted.

As shown in FIG. 4, the sprayer unit 120 according to this embodiment further includes the cladding 123 for sprayer surrounding the outer surface of the spray body 121. In this embodiment, the refractive index of the cladding 123 for sprayer is lower than the refractive index of the spray body 121, to focus the laser beam to the spray body 121 and direct it toward the metal foil 122.

The cladding 123 for sprayer is formed by applying a low-refractive-index optical fiber coating agent to the outer surface of the spray body 121. In this embodiment, the low-refractive-index optical fiber coating agent may include Norland Optical Adhesive (NOA) 1348.

Meanwhile, as shown in FIG. 5, the method for manufacturing the optical atomizer according to this embodiment further includes a cladding formation step (S230) performed after the metal foil deposition step (S220) to form the cladding 123 for sprayer surrounding the outer surface of the spray body 121.

In the cladding formation step (S230), the low-refractive-index optical fiber coating agent is applied to the outer surface of the spray body 121 to form the cladding 123 for sprayer.

Because the optical atomizer according to this embodiment has the cladding 123 for sprayer surrounding the outer surface of the spray body 121, it may be possible to transmit the laser beam to the metal foil 122 more efficiently.

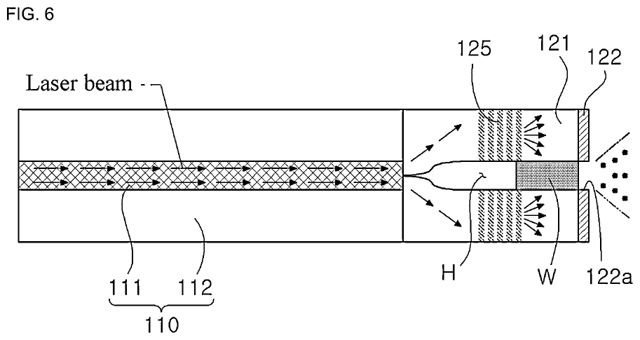

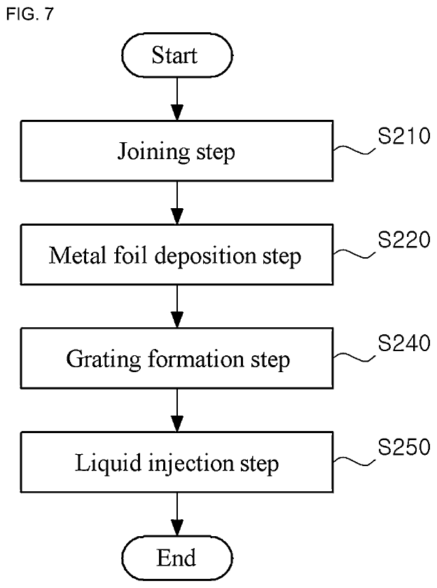

FIG. 6 is a cross-sectional view showing the optical atomizer according to a third embodiment of the present disclosure, and FIG. 7 is a flowchart showing the method for manufacturing the optical atomizer of FIG. 6.

When compared with the first embodiment, this embodiment further includes a long-period fiber grating 125 but has the same components as the first embodiment of FIGS. 1 to 3, and hereinafter the description of the same components is omitted.

As shown in FIG. 6, the spray body 121 according to this embodiment includes the long-period fiber grating 125 to excite the laser beam transmitted from the laser transmitter unit 110 in a cladding mode.

The long-period fiber grating 125 excites the Gaussian laser beam transmitted from the laser transmitter unit 110 in the cladding mode.

The long-period fiber grating 125 is formed by ultraviolet laser beam irradiation to the spray body 121. The structure of the long-period fiber grating depicted in FIG. 6 effectively works in the laser transmitter unit 110 of a single-mode optical fiber. Meanwhile, in case where the laser transmitter unit 110 is replaced with a multiple-mode optical fiber, a similar effect is exhibited without the long-period fiber grating structure since the laser beam already excited in the cladding mode is transmitted to the spray body 121.

Meanwhile, as shown in FIG. 7, the method for manufacturing the optical atomizer according to this embodiment includes a grating formation step (S240) performed after the metal foil deposition step (S220) to form the long-period fiber grating 125 in the spray body 121 to excite the laser beam transmitted from the laser transmitter unit 110 in the cladding mode.

In the grating formation step (S240), the long-period fiber grating 125 is formed by ultraviolet laser beam irradiation to the spray body 121.

Because the optical atomizer according to this embodiment includes the spray body 121 having the long-period fiber grating 125 to excite the laser beam in the cladding mode, it may be possible to transmit the laser beam to the metal foil 122 more efficiently.

While this embodiment has been hereinabove described with reference to the accompanying drawings, the scope of protection of this embodiment is not limited to the accompanying drawings and the foregoing description.

The present disclosure is not limited to the disclosed embodiments, and it is obvious to those skilled in the art that a variety of modifications and change may be made thereto without departing from the scope and spirit of the present disclosure. Accordingly, it should be understood that such modifications or variations fall within the scope of protection of the present disclosure.

[Detailed Description of Main Elements]

| | |
|---|---|
| 110: Laser transmitter unit | 120: Sprayer unit |
| 121: Spray body | 122: Metal foil |
| 122a: Spray hole | 123: Cladding for sprayer |
| 125: Long-period fiber grating | H: Hollow groove |
| W: Liquid for spraying | |

What is claimed is:

1. An optical atomizer comprising:
a laser transmitter unit configured to transmit a laser beam oscillated by a laser oscillator to one side; and
a sprayer unit connected to the laser transmitter unit, and configured to heat a liquid for spraying received in the sprayer unit by the laser beam transmitted from the laser transmitter unit and spray the evaporated liquid for spraying to outside.

2. The optical atomizer according to claim 1, wherein the sprayer unit includes:
a spray body having an end portion joined to the laser transmitter unit, and an opposite end portion having an opening of a hollow groove in which the liquid for spraying is received; and
a metal foil deposited on the opposite end portion of the spray body, and having a spray hole in communication with the hollow groove.

3. The optical atomizer according to claim 2, wherein the spray body is made of a coreless Hollow Optical Fiber (HOF).

4. The optical atomizer according to claim 1, wherein the liquid for spraying includes a high viscosity liquid.

5. The optical atomizer according to claim 1, wherein the liquid for spraying includes any one selected from triethylene glycol, glycerol and oleic acid.

6. The optical atomizer according to claim 2, wherein the metal foil is deposited on the spray body by a sputtering process.

7. The optical atomizer according to claim 2, wherein the metal foil is made of titanium.

8. The optical atomizer according to claim 2, wherein a bottom area of the hollow groove is formed with a smaller diameter toward the laser transmitter unit.

9. The optical atomizer according to claim 2, wherein the sprayer unit further includes a cladding for sprayer surrounding an outer surface of the spray body.

10. The optical atomizer according to claim 9, wherein the cladding for sprayer is formed by applying a low-refractive-index optical fiber coating agent to the outer surface of the spray body.

11. The optical atomizer according to claim 2, wherein the spray body has a long-period fiber grating to excite the laser beam transmitted from the laser transmitter unit in a cladding mode.

12. The optical atomizer according to claim 11, wherein the long-period fiber grating is formed by ultraviolet laser beam irradiation to the spray body.

13. A spraying method using an optical atomizer, comprising:
a laser transmission step of transmitting a laser beam oscillated by a laser oscillator to a spray body having a hollow groove in which a liquid for spraying is received;
a heating and spraying step of heating a metal foil deposited on the spray body and having a spray hole in communication with the hollow groove by the transmitted laser beam, and spraying the liquid for spraying evaporated by the heated metal foil out of the hollow groove; and
a condensation step of condensing the evaporated liquid for spraying sprayed out of the hollow groove into droplets.

14. The spraying method using the optical atomizer according to claim 13,
wherein the metal foil is heated by a photothermal effect in the heating and spraying step.

15. The spraying method using the optical atomizer according to claim 13, wherein the liquid for spraying includes any one selected from triethylene glycol, glycerol and oleic acid, and
wherein the metal foil is made of titanium.

16. A method for manufacturing an optical atomizer, comprising:
a joining step of joining a spray body having a hollow groove to a laser transmitter unit configured to transmit a laser beam oscillated by a laser oscillator to one side;
a metal foil deposition step of depositing a metal foil having a spray hole in communication with the hollow groove on the spray body; and
a liquid injection step of injecting a liquid for spraying into the hollow groove.

17. The method for manufacturing the optical atomizer according to claim 16, wherein the metal foil is deposited on the spray body by a sputtering process.

18. The method for manufacturing the optical atomizer according to claim 16, wherein the metal foil is made of titanium.

19. The method for manufacturing the optical atomizer according to claim 16, wherein the liquid for spraying is injected into the hollow groove through a capillary phenomenon in the liquid injection step.

20. The method for manufacturing the optical atomizer according to claim 16, further comprising:
after the metal foil deposition step,
a cladding formation step of forming a cladding for sprayer surrounding an outer surface of the spray body.

21. The method for manufacturing the optical atomizer according to claim 20, wherein the cladding formation step comprises applying a low-refractive-index optical fiber coating agent to an outer surface of the spray body to form the cladding for sprayer.

22. The method for manufacturing the optical atomizer according to claim 16, further comprising:
after the metal foil deposition step,
a grating formation step of forming a long-period fiber grating in the spray body to excite the laser beam transmitted from the laser transmitter unit in a cladding mode.

23. The method for manufacturing the optical atomizer according to claim 22, wherein the grating formation step comprises forming the long-period fiber grating by ultraviolet laser beam irradiation to the spray body.
